(19) United States
(12) Patent Application Publication
(10) Pub. No.: US 2012/0259023 A1
(43) Pub. Date: Oct. 11, 2012

(54) NOVEL PLANT GUM-MODIFIED FOOD STARCH CONJUGATE AS EMULSIFYING AGENT FOR FAT SOLUBLE INGREDIENTS

(76) Inventors: Andrea Hitzfeld, Grenzach-Wyhlen (DE); Bruno H. Leuenberger, Rheinfelden (CH); Paul Steeman, Spaubeek (NL); Olivia Vidoni, Saint Louis (FR)

(21) Appl. No.: 13/499,751

(22) PCT Filed: Oct. 1, 2010
(86) PCT No.: PCT/EP2010/064618

§ 371 Date: Jun. 29, 2012

(30) Foreign Application Priority Data
Oct. 2, 2009 (EP) 09172051.6

(51) Int. Cl. C08B 37/00; A61K 8/73; A23L 1/0522; A23L 1/302; A23L 2/52; A23D 9/00; A23L 1/275; A23K 1/16; A23L 1/30; A23L 2/58; A61K 47/36; A23L 1/303

(52) U.S. Cl. 514/778; 536/102; 426/661; 426/72; 426/73; 426/601; 426/540; 426/648; 426/590

(57) ABSTRACT

The present invention relates to novel conjugates based on plant gums and modified food starch, a process for the manufacture thereof, as well as to compositions containing active ingredients, preferably fat-soluble active ingredients, and/or colorants in a matrix based on these conjugates, and to a process for preparing these compositions.

The present invention relates to conjugates based on plant gums and modified food starch, a process for the manufacture thereof, as well as to compositions containing active ingredients, preferably fat-soluble active ingredients, and/or colorants in a matrix based on these conjugates, and to a process for preparing these compositions.

The present invention further relates to the use of the compositions of this invention for the enrichment, fortification and/or coloration of food beverages, animal feed and/or cosmetics or pharmaceutical compositions.

More particularly, the present invention relates to compositions containing one or more plant gums, one or more modified food starches and one or more fat-soluble active ingredients and/or a colorant, especially a carotenoid, to a process for preparing these compositions and the use of these compositions as additives for the enrichment fortification and/or coloration of food beverages, animal feed and/or cosmetics or pharmaceutical compositions; furthermore the present invention relates to food, beverages, animal feed, cosmetics or pharmaceutical compositions containing such compositions.

Compositions to enrich fortify or color food, beverages, animal feed or cosmetics which contain fat-soluble active ingredients, for example beta-carotene, are known in the art. Beta-Carotene is a preferable colorant compound due to its intense and for the above-mentioned applications very pleasing orange color. Since the final compositions are often aqueous compositions such as beverages, additional compounds have to be added to the compositions for the enrichment, fortification and/or coloration to avoid separation of fat (oil) phases from the product, which would render the corresponding product unacceptable.

Therefore, fat-soluble active ingredients are often combined with auxiliary compounds, such as starches or fish gelatin, in order to prevent phase separation in the final aqueous composition. Those auxiliary compounds, however, often have a negative influence on the color properties and/or the nutritional properties of the final products. It is therefore desired to develop new compositions of fat-soluble active ingredients, which contain improved auxiliary compounds, which have very good properties referring to color of the composition, taste, emulsification, emulsion stability, and/or film forming ability. Moreover, there is an increased need in the industry to develop such auxiliary compounds based on raw materials of natural origin to replace gelatin.

Gum acacia (also called gum arabic), a natural hydrocolloid is widely used as an emulsifier/stabilizer in beverage emulsions. It is highly water soluble (up to 50% in weight) and its aqueous solution provides, emulsion stability, encapsulation and film forming ability. Gum acacia is obtained as sticky exudates from the stems and branches of acacia trees when they are subjected to stress. The gum is collected from trees that belong to the genus Acacia of the Leguminosae family and that grow in several countries in the Sahara region of Africa. The gum is a natural macromolecule belonging to the class of glycoproteins. It comprises a polypeptide chain mainly composed of the amino acids: hydroxyproline, serine, proline bonded to an arabinogalactan polysaccharide chain consisting of rhamnose and glucoronic acid end units and four sugars (L-arabinose, L-rhamnose, D-galactose, D-glucuronicacid) (Idris et al, Food Hydrocolloids, Part I to III, 12, 1998, 379-388).

Acacia senegal (A. senegal) is the most common commercial gum arabic, and also the most available gum. The other gum available, Acacia seyal (A. seyal), has lower emulsifying properties than Acacia senegal, but is cheaper. However, uneven performances of the gum may arise among different lots of plant gum because of dissimilar functionality related to species, geographical location, nature of the soil, climate, and age of the trees.

WO2008110225 discloses a heat treatment of gum acacia at a temperature between 100° C. and 115° C. for 1 to 38 hours to improve the emulsifying properties of carotenoid compositions. However, performance related to the color intensity value and the stability of carotenoids prepared using such gum acacia conjugates remains extremely variable.

The term “Modified food starch” as used herein relates to modified starches that are made from starches substituted by known chemical methods with hydrophobic moieties. For example starch may be treated with cyclic dicarboxylic acid anhydrides such as succinic and/or glutaric anhydrides, substituted with an alkyl or alkenyl hydrocarbon group. Modified food starch is commonly used in the prior art as matrix for compositions containing fat-soluble active ingredients and/or colorants, but lack to demonstrate stability over time when used in beverage preparations.

On the other hand, protein-polysaccharide conjugates are also known in the art (EP1776873) to improve emulsifying properties of proteins, especially through oil droplet size reduction and emulsion stabilization. The conjugates are adsorbed at the interface together with unreacted protein constituents, enhancing steric stabilization forces of oil droplets. However, the emulsion stability of the protein-polysaccharide conjugate around isoelectric point remains inadequate for use in food and beverage applications and protein emulsifiers in food and beverage applications remain an issue.

There is still a need for new emulsifiers in food and beverage applications to prepare compositions of fat-soluble active ingredients for the enrichment, fortification and/or coloration of food, beverages, animal feed, cosmetics or pharmaceutical compositions which do not show the above-mentioned problems.

It was therefore an objective of the present invention to identify a new emulsifier and to provide compositions of fat-soluble active ingredients having the desired properties as indicated above, e.g. improved color intensity and color stability, very good properties referring to optical clarity and emulsion stability (wherever applicable) without using any product of animal origin. It was also an objective of the invention to improve the process for the preparation of compositions of fat-soluble active ingredients for example by using a new emulsifier.

It has surprisingly been found that conjugates based on plant gums and modified food starch fulfilled the need in industry.

In the present context, the term “conjugate” refers to high molecular weight polymeric molecules covalently linked through Maillard type reaction often requiring heating. They usually result from a reaction between a reactive carbonyl group of a sugar moiety with a nucleophilic amino group of an amino acid. A conjugate may generally be characterized by the fact that its molecular weight is higher than the molecular weight of the starting non conjugated molecules and by the fact that it forms a stable polymeric chemical entity.

The first object of the present invention is a novel plant gum—modified food starch conjugate consisting of

a) one or more plant gum(s);
b) one or more modified food starch
wherein a 30 wt % solution in water of said isolated plant gum—modified food starch conjugate at 25° C. has a phase angle of less than 85 degrees at a value of the dynamic modulus of 1 Pa, as determined with oscillatory rheological measurements.

Gum acacia and/or gum ghatti can be used as plant gums according to the present invention. The most preferred plant gum is gum acacia comprising for example gums from the species Acacia senegal, and/or Acacia seyal produced in any African country. It is also possible to use plant gums that have been subjected to a treatment such as purification treatment, desalting treatment, pulverization, or spray drying without any restriction in their water content. Furthermore, plant gums procured in the forms of blocks, beads, coarse pulverizates, granules, pellets, and powders (including spray dried powder) can be used without any preference.

In a preferred embodiment, commercially available gum from Acacia senegal is used in the present invention.

According to the present invention, the preferred modified starch is starch sodium octenyl succinate (“OSA-starch”). OSA-starches may contain further hydrocolloids, such as starch, maltodextrin, carbohydrates, gum, corn syrup etc. and optionally any typical emulsifier (as co-emulsifier), such as mono- and diglycerides of fatty acids, polyglycerol esters of fatty acids, lecithins, sorbitan monostearate, and plant fibre or sugar. OSA-starches are commercially available e.g. from National Starch under the trade names HiCap 100, Capsul, Capsul HS, Purity Gum 2000, UNI-PURE, HYLON VII; from Roquette Freres; from CereStar under the tradename C*EmCap or from Tate & Lyle.

The mean molecular weight used to characterize the plant gum—modified food starch conjugate in expressed in gram per mol and is measured by asymmetric flow field flow fractionation. Asymmetric Field Flow Field Fractionation (A4F) is a one-phase chromatography technique. High-resolution separation is achieved within a very thin flow against which a perpendicular force field is applied.

The flow and sample are confined within a channel consisting of two plates that are separated by a spacer foil. Sample constituents elute and are separated according to their size (diffusion driven by Brownian motion) and are monitored by an array of detectors. The smallest particles are transported much more rapidly along the channel than the largest ones. Elution time of the smallest particles is faster than the one of the largest; this is the main difference with Size Exclusion/Gel Permeation Chromatography (SEC/GPC) in which one the largest molecules elute first (IMM Beauvais, France, and Comprehensive Polymer Science, Vol 1, page 279).

Plant gum—modified food starch conjugates according to the present invention may be characterized by the fact that their mean molecular weight is at least 1.2 fold higher when compared to the mean molecular weight of the starting plant gum material; preferably their mean molecular weight is at least increased by a factor 2; most preferably at least increased by a factor 3. As an example, gum acacia has a mean molecular weight of around 3 million g/mol, while the mean molecular weight of the plant gum—modified food starch conjugate produced according to the present invention is about 10 million g/mol.

Plant gum—modified food starch conjugates according to the present invention may be further characterized by their rheological properties in aqueous solution (30% solution of the isolated conjugate). Steady state and oscillatory (dynamic) viscosity measurements may be carried out with concentric cylinder geometry (e.g. Mooney type, or double gap geometry). Dynamic rheology measurements have confirmed an increase of the molecular weight and a broadening of molecular weight distribution of plant gums modified food starch conjugates filtrated through a 500 K Dalton membrane (HOLLOW FIBER—GE Healthcare).

Aqueous solutions (33% w/w) of plant gum—modified food starch conjugates of the present invention, filtrated through a 500 k Dalton membrane (HOLLOW FIBER—GE Healthcare), present an increased viscosity at 25° C. by a factor of at least 1.2 when compared to non conjugated gums.

Additionally, the molecular structure of the plant gum—modified food starch may be characterized at 25° C. in a 30 wt % water solution by its phase angle as described in example 5. Plant gum—modified food starch conjugates of the present invention may be characterized by the fact that said conjugates have a phase angle of less than 85 degrees at a dynamic modulus of 1 Pa. More preferably, the phase angle of these conjugates at a dynamic modulus of 1 Pa is less than 82.5 degrees, even more preferably less than 80 degrees and most preferably less than 75 degrees.

The form of the plant gum—modified food starch conjugates according to the present invention is not limited and includes for example blocks, beads, coarse pulverizates, granules, pellets or powders.

It was also an objective of the present invention to provide compositions of fat-soluble active ingredients having the desired properties as indicated above, e.g. very good properties referring to optical clarity and emulsion stability and/or an improved color intensity and color stability (wherever applicable).

An additional object of the present invention is a composition comprising a) 10 to 70 weight-% of plant gum—modified food starch conjugate as described above; and b) 0.1 to 60 weight-% of one or more fat-soluble active ingredient(s), each based on the weight of the total composition in dry matter.

The composition according to this invention preferably comprises 15 to 60 weight-% of plant gum—modified food starch conjugate based on the total composition in dry matter. and/or 0.1 to 30 weight-%, further preferred between 0.5 and 20 weight-%, most preferred between 0.5 and 15 weight-% of one or more fat soluble ingredient, each based on the total composition in dry matter.

As used herein, the term “fat-soluble active ingredient” refers to vitamins selected from the group consisting of vitamin A, D, E, K and derivatives thereof; polyunsaturated fatty acids and esters thereof; lipophilic active ingredients; carotenoids; and flavoring or aroma substances as well as mixtures thereof.

Polyunsaturated fatty acids (PUFAs), which are suitable according to the present invention, are mono- or polyunsaturated carboxylic acids having preferably 16 to 24 carbon atoms and, in particular, 1 to 6 double bonds, preferably having 4 or 5 or 6 double bonds.

The unsaturated fatty acids can belong both to the n-6 series and to the n-3 series. Preferred examples of n-3 polyunsaturated acids are eicosapenta-5,8,11,14,17-enoic acid and docosahexa-4,7,10,13,16,19-enoic acid; preferred examples of a n-6 polyunsaturated acid are arachidonic acid and gamma linolenic acid.

Preferred derivatives of the polyunsaturated fatty acids are their esters, for example glycerides and, in particular, triglycerides; particularly preferably the ethyl esters. Triglycerides of n-3 and n-6 polyunsaturated fatty acids are especially preferred.

The triglycerides can contain 3 uniform unsaturated fatty acids or 2 or 3 different unsaturated fatty acids. They may also partly contain saturated fatty acids.

When the derivatives are triglycerides, normally three different n-3 polyunsaturated fatty acids are esterified with glycerin. In one preferred embodiment of the present invention triglycerides are used, whereby 30% of the fatty acid part are n-3 fatty acids and of these 25% are long-chain polyunsaturated fatty acids. In a further preferred embodiment commercially available ROPUFA® ‘30’ n-3 Food Oil (DSM Nutritional Products Ltd, Kaiseraugst, Switzerland) is used.

In another preferred embodiment of the present invention, the PUFA ester is ROPUFA® ‘75’ n-3 EE. ROPUFA ‘75’ n-3 EE is refined marine oil in form of an ethyl ester with minimum content of 72% n-3 fatty acid ethyl ester. It is stabilized with mixed tocopherols, ascorbyl palmitate, citric acid and contains rosemary extract.

In another preferred embodiment of the present invention the PUFA ester is ROPUFA® ‘10’ n-6 Oil, a refined evening primrose oil with minimum 9% gamma linolenic acid which is stabilized DL-alpha-tocopherol and ascorbyl palmitate.

According to the present invention it can be advantageous to use naturally occurring oils (one or more components) containing triglycerides of polyunsaturated fatty acids, for example marine oils (fish oils) and/or plant oils, but also oils extracted from fermented biomass or genetically modified plants

Preferred oils which comprise triglycerides of polyunsaturated fatty acids are olive oil, sunflower seed oil, evening primrose seed oil, borage oil, grape seed oil, soybean oil, groundnut oil, wheat germ oil, pumpkin seed oil, walnut oil, sesame seed oil, rapeseed oil (canola), blackcurrant seed oil, kiwifruit seed oil, oil from specific fungi and fish oils.

Preferred examples for polyunsaturated fatty acids are e.g. linoleic acid, linolenic acid, arachidonic acid, docosahexaenic acid, eicosapentaenic acid and the like.

According to the present invention preferred lipophilic active ingredients are resveratrol; ligusticum; ubiquinones and/or ubiquinols (one or more components) selected from coenzyme Q 10 (also referred to as “CoQ10”), coenzyme Q 9, and/or their reduced forms (the corresponding ubiquinols); genistein and/or alpha-lipoic acid.

According to the present invention, preferred fat-soluble active ingredients are carotenoids, especially beta-carotene, lycopene, lutein, bixin, astaxanthin, apocarotenal, beta-apo-8′-carotenal, beta-apo-12′-carotenal, canthaxanthin, cryptoxanthin, citranaxanthin and zeaxanthin. Most preferred is beta-carotene.

Additionally, the compositions of the present invention may (further) contain one or more excipients and/or adjuvants selected from the group consisting of monosaccharides, disaccharides, oligosaccharides and polysaccharides, water-soluble antioxidants and fat-soluble antioxidants.

Examples of mono- and disaccharides which may be present in the compositions of the present invention are sucrose, invert sugar, xylose, glucose, fructose, lactose, maltose, saccharose and sugar alcohols.

Examples of the oligo- and polysaccharides are starch, starch hydrolysates, e.g. dextrins and maltodextrins, especially those having the range of 5 to 65 dextrose equivalents (DE), and glucose syrup, especially such having the range of 20 to 95 DE. The term “dextrose equivalent” (DE) denotes the degree of hydrolysis and is a measure of the amount of reducing sugar calculated as D-glucose based on dry weight; the scale is based on native starch having a DE close to 0 and glucose having a DE of 100. Preferably, maltodextrin is used in the composition according to the invention.

The water-soluble antioxidant may be for example ascorbic acid or a salt thereof, preferably sodium ascorbate, water-soluble polyphenols such as hydroxy tyrosol and oleuropein, aglycone, epigallo catechin gallate (EGCG) or extracts of rosemary or olives.

The fat-soluble antioxidant may be for example a tocopherol, e.g. dl-α-tocopherol (i.e. synthetic tocopherol), d-α-tocopherol (i.e. natural tocopherol), β- or γ-tocopherol, or a mixture of two or more of these; butylated hydroxytoluene (BHT); butylated hydroxyanisole (BHA); ethoxyquin, propyl gallate; tert.-Butyl-hydroxyquinoline; or 6-ethoxy-1,2-dihydroxy-2,2,4-trimethylquinoline (EMQ), or an ascorbic acid ester of a fatty acid, preferably ascorbyl palmitate or stearate.

The composition according to the invention preferably comprises less than 30%, further preferred less than 10 weight-%, further preferred less than 3 weight-% oil (based on the weight of the total composition in dry matter). Most preferably the composition does not comprise any oil.

The expression “oil” as used in this context comprises any trigylcerides or any other oil (e.g. terpene), which is suitable for the desired use of the composition. The triglyceride is suitably a vegetable oil or fat, preferably corn oil, sunflower oil, soybean oil, safflower oil, rapeseed oil, peanut oil, palm oil, palm kernel oil, cotton seed oil, orange oil, limonene, olive oil or coconut oil.

Solid compositions may in addition contain an anti-caking agent, such as silicic acid or tricalcium phosphate and the like, and up to 10 weight-%, preferably 0.1 to 5 weight-%, each (based on the weight of the total composition in dry matter).

Additionally, the composition can comprise water.

In a further aspect of the invention, the manufacture of the plant gum—modified food starch conjugate comprises the following steps:
I) Suspending separately both plant gum and modified food starch into water;
II) Optionally removing non dissolved material from the suspended modified food starch;
III) Mixing the aqueous solution of plant gum of step I) to the aqueous solution of modified food starch prepared as in step I) and optionally II) in a ratio 1:1 to 20:1 and stirring the resulting mixture for about one hour;
IV) Optionally, incubating the mixture of step III) 1 to 24 hours at room temperature;
V) Drying the mixture of step IV) to remove water and to produce a powder;
VI) Heating the resulting powder of step V) between 50° and 150° C. for 1 to 72 hours to form the plant gum—modified food starch conjugate.

The plant gum—modified food starch conjugate of step VI) may be used as such, dried in e.g. a desiccator for later use, or optionally further purified (e.g. for characterization of the conjugate) according to the following steps:
VII) Optionally, suspending plant gum-modified food starches conjugates of step VI) in water;
VIII) Optionally, diafiltration of the aqueous solution through a 500 k Dalton membrane (HOLLOW FIBER—GE Healthcare) until the volume diluted (Vd) was at least greater or equal to 4. (Vd=volume of liquid permeated/initial feed volume).
IX) Drying the mixture of step VIII) to remove water and to produce a powder.

This process is exemplified in Example 1. The preferred aqueous solution of plant gum contains 8% w/w of gum acacia, but is not limited to this concentration. The preferred aqueous solution of modified food starch is a 30% w/w solution, but is not limited to this concentration. The preferred weight ratio of plant gums to modified food starch is chosen within the range 1:1 to 20:1, further preferred weight ratio is chosen within the range 3:2 to 4:1, further preferred weight ratio is chosen within the range 13:7 to 3:1, and further preferred weight ratio is chosen within the range 2:1 to 5:2.

Drying of the mixture step V) can be performed by spray drying in order to keep the water content below 8%.

Step VI) is best performed by heating between 100 and 120° C. for 12 to 72 hours, but temperature can range from 50 to 150° C. and time from 1 to 72 hours.

In another preferred embodiment, the present invention relates to a process for the manufacture of a composition comprising a plant gum—modified food starch conjugate as indicated above and further comprising one or more fat-soluble ingredient(s). This process comprises the following steps:
I) dispersing an appropriate amount of plant gum—modified food starch conjugate obtainable by a process as described above in an appropriate amount of water
II) adding one or more fat soluble active ingredient(s) to the solution produced in step I)
III) emulsifying the mixture of step II) with a conventional emulsification process known to the person skilled in the art.
IV) optionally drying the emulsion of step III)

This process is performed according to a process already described in: WO2008110225

The present invention is also directed to the use of compositions as described above for the enrichment, fortification and/or coloration of food, beverages, animal feed and/or cosmetics, preferably for the enrichment, fortification and/or coloration of beverages.

Other aspects of the invention are food, beverages, animal feed, cosmetics containing a composition as described above.

Beverages wherein the product forms of the present invention can be used as a colorant or an additive ingredient can be carbonated beverages e.g., flavored seltzer waters, soft drinks or mineral drinks, as well as non-carbonated beverages e.g. flavored waters, fruit juices, fruit punches and concentrated forms of these beverages. They may be based on natural fruit or vegetable juices or on artificial flavors. Also included are alcoholic beverages and instant beverage powders. Besides, sugar containing beverages diet beverages with non-caloric and artificial sweeteners are also included.

Further, dairy products, obtained from natural sources or synthetic, are within the scope of the food products wherein the product forms of the present invention can be used as a colorant or as an additive ingredient. Typical examples of such products are milk drinks, ice cream, cheese, yogurt and the like. Milk replacing products such as soymilk drinks and tofu products are also comprised within this range of application.

Also included are sweets which contain the product forms of the present invention as a colorant or as an additive ingredient, such as confectionery products, candies, gums, desserts, e.g. ice cream, jellies, puddings, instant pudding powders and the like.

Also included are cereals, snacks, cookies, pasta, soups and sauces, mayonnaise, salad dressings and the like which contain the product forms of the present invention as a colorant or an additive ingredient. Furthermore, fruit preparations used for dairy and cereals are also included.

The final concentration of the one or more fat-soluble active ingredients, preferred carotenoids, especially beta-carotene, which is added via the compositions of the present invention to the food products may preferably be from 0.1 to 50 ppm, particularly from 1 to 30 ppm, more preferred 3 to 20 ppm, e.g. about 6 ppm, based on the total weight of the food composition and depending on the particular food product to be colored or fortified and the intended grade of coloration or fortification.

The food compositions of this invention are preferably obtained by adding to a food product the fat-soluble active ingredient in the form of a composition of this invention. For coloration or fortification of a food or a pharmaceutical product a composition of this invention can be used according to methods per se known for the application of water dispersible solid product forms.

In general the composition may be added either as an aqueous concentrated solution, a dry powder or a pre-blend with other suitable food ingredients according to the specific application. Mixing can be done e.g. using a dry powder blender, a low shear mixer, a high-pressure homogenizer or a high shear mixer depending on the formulation of the final application. As will be readily apparent such technicalities are within the skill of the expert.

The present invention is further illustrated by the following examples, which are not intended to be limiting.

### EXAMPLES

### Example 1

### Preparation of the Gum Acacia-OSA Starch Conjugate

To an aqueous solution of gum acacia (8% w/w) was added an aqueous solution of OSA Starch (30% w/w). The mixture was mixed during one hour at room temperature, and let undisturbed overnight for complete hydration. The day after, product was spray-dried (Niro-Mobile MinorTN “2000”-Tᵢₙ: 180° C. Tₒᵤₜ: 75° C.). The powder collected was then transferred in two liter beakers, heated on at 110° C. in an oven for 20 hours and stored in a desiccator.

The following conjugates were prepared:

TABLE 1

| | Composition of samples | |
|---|---|---|
| Sample name | Gum acacia content (%) | OSA Starch content (%) |
| GA-OSA 1/1 | 50 | 50 |
| GA-OSA 7/3 | 70 | 30 |
| GA-OSA 9/1 | 90 | 10 |
| GA-OSA 19/1 | 95 | 5 |

### Example 2

### Purification of the Gum Acacia-OSA Starch Conjugate

An aqueous solution of each gum acacia-OSA starch conjugate (8 to 10% w/w) was filtered with continuous diafiltration through a hollow fibres membrane with a molecular weight cut-off of 500 KDa (GE Healthcare-Type UFP-500-E-6A) with a TMP of 1 bar. The process was stopped when permeate's sugar level was too low to be detected (UV measurements). The retentate solution was finally spray-dried (Niro-Mobile Minor TN “2000”-Tᵢₙ: 180° C. Tₒᵤₜ: 75° C.), and the collected product stored and used without any further modifications.

### Example 3

### Characterization of the gum acacia-OSA starch conjugate by measurement of the molecular weight through asymmetric flow field flow fractionation as performed by IMM-Beauvais, France.

### Technical Details:

Elution buffer: Water Ultra High Quality (UHQ—bi-demineralized, filtered at 0.2 μm, UV treated)
On-lined filter type: 0.1 μm, modified cellulose
Fractionation system: WYATT ECLIPSE 3
Multi angle laser light scattering measurements: 658 nm
Refractive index measurements: 658 nm, do/dc=0.19 ml/g
UV Agilent 1200 serie: 280 nm
Ultra filtration membrane used: regenerated cellulose, cut-off 10 kDa
Thickness of the spacer: 250 μm
Length of fractionation cell: 13 cm (SCELL)

Sample treatment before injection: sample was solubilized in UHQ Water, during a night, at room temperature. Sample was then directly injected without any filtration.

TABLE 2
Mean molecular weight of plant gum-OSA starch conjugates
as measured by asymmetric flow filed flow fractionation.

| | | Mean molecular |
|---|---|---|
| Sample | Recovery (%) | weight (g/mol) |
| Gum acacia | 93.7 | 3.0 × 10⁶ |
| Gum acacia-OSA starch 1:1 | 89.8 | 1.2 × 10⁷ |
| Gum acacia/OSA starch 7:3 | 91.2 | 1.1 × 10⁷ |
| gum acacia/OSA starch 9:1 | 92.3 | 8.2 × 10⁶ |
| gum acacia/OSA starch 19:1 | 92.6 | 8.7 × 10⁶ |

A high recovery value is an indication of a good solubility of the product prior injection and a good separation and detection on the column. It can be linked to a good reproducibility of the methodology.

### Example 4

### Characterization of the Gum Acacia-OSA Starch Conjugate by Viscosity

Viscosity measurements of aqueous solutions of each gum acacia-OSA starch conjugate (33% w/w) have been performed with concentric cylinder geometry (Mooney) using a TA Instruments AR-G2 controlled stress rheometer.

Results are displayed in Table 3: The viscosity values of each entity have been determined at 25° C. with a shear rate of 20s⁻¹.

TABLE 3
Summary of viscosity measurements performed on non isolated
gum acacia-OSA starch conjugates, or with isolated material
according to the optional steps VII) to IX).

| Components | | η(Pa · s) @ 25° C. |
|---|---|---|
| Gum acacia | | 0.47 |
| Gum acacia cross-linked 48 hours | | 0.66 |
| OSA starch | | 0.06 |
| OSA starch cross-linked 48 hours | | 0.04 |
| Gum acacia-OSA starch conjugates | composition | |
| Non isolated | 1:1 | 0.24 |
| | 7:3 | 0.33 |
| | 9:1 | 0.68 |
| | 19:1 | 0.63 |
| Isolated | 1:1 | 0.64 |
| | 7:3 | 1.70 |
| | 9:1 | 0.94 |
| | 19:1 | 1.4 |

### Example 5

### Rheological Measurements in Aqueous Solution of Gum Acacia-OSA Starch Conjugate

The dynamic rheological properties of 30 wt % solutions of the isolated conjugates in water were determined at 25° C. Oscillatory measurements have been carried out with a TA Instruments AR-G2 rheometer equipped with a double gap geometry. A so-called frequency sweep was performed with 5 frequencies per decade, between 628 and 0.628 rad/s in decreasing order. The instrument was run in the controlled strain mode with the amplitude of the applied deformation fixed to 10%.

The instrument produces data for the dynamic rheological properties which may be represented in various formats that can be easily inter-converted. Starting from the frequency dependent storage modulus G′(ω) and the loss modulus G″ (ω), the following equations hold (J. D. Ferry, Viscoelastic Properties of Polymers', John Wiley & Sons, (1980) 3rd Edition):

The dynamic modulus Gd(ω)=(G′(ω)²+G″(ω)²)^{0.5}

The phase angle δ=arctan(G″(ω)/G′(ω))

The dynamic viscosity Etad(ω)=Gd(ω)/ω

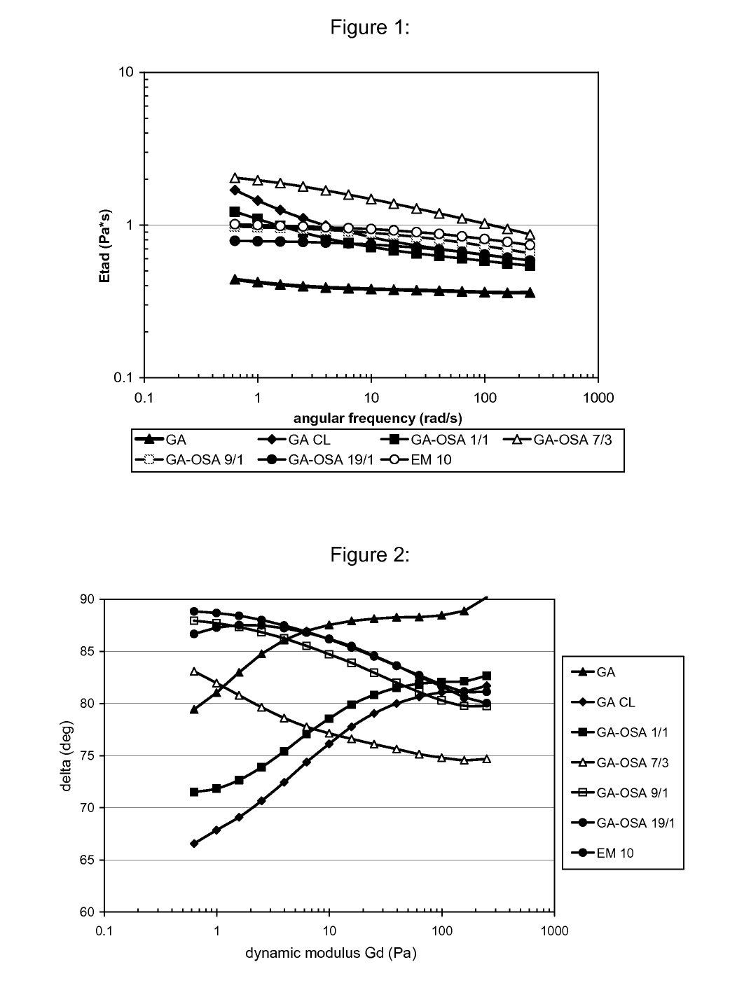

FIG. 1 shows the dynamic viscosity Etad versus the angular frequency w. Data obtained at frequencies higher than 250 rad/s were considered to be unreliable due to the high inertia of the double gap geometry used for the dynamic measurements.

FIG. 1: The dynamic viscosity of the Gum Arabic materials as a function of the frequency. GA=Unmodified gum arabic; GA CL=gum Arabic cross linked for 48 hours; GA-OSA 1/1=gum Arabic-OSA starch conjugate prepared in a 1/1 ratio; GA-OSA 7/3=gum Arabic-OSA starch conjugate prepared in a 7/3 ratio; GA-OSA 9/1=gum Arabic-OSA starch conjugate prepared in a 9/1 ratio; GA-OSA 19/1=gum Arabic-OSA starch conjugate prepared in a 19/1 ratio; EM 10=Super Gum EM10″ from San -Ei Gen F.F.I

The rheological behavior of the materials is mainly that of a Newtonian liquid (showing an approximately frequency independent viscosity). However, a few materials show a clear upswing of the viscosity at frequencies below 10 rad/s, which is attributed to the presence of an elastic/gel-like fraction.

It is common practice to use the solution viscosity as a molecular weight indicator for the dissolved polymer. In this case, we defined the dynamic viscosity (Etad) at an angular frequency of 10 rad/s as molar mass indicator (Mwt) for the dissolved Gum Acacia polymer. This choice of the frequency excludes an influence of the gel fraction if present to the molar mass indicator. This results in a ranking for the molecular weight of the materials:

Mwt GA<GA-OSA1/1≦GA-OSA 19/1<GA CL<GA-OSA 9/1<EM10<GA-OSA 7/3

In order to get more detailed information on the molecular structure of the materials a plot of the phase angle δ versus the dynamic modulus Gd(ω) is shown in FIG. 2. Again, data measured at frequencies higher that 250 rad/s were excluded from the analysis.

FIG. 2: The phase angle versus the dynamic modulus for the various Gum Arabic materials. GA=Unmodified gum arabic; GA CL=gum Arabic cross linked for 48 hours; GA-OSA 1/1=gum Arabic-OSA starch conjugate prepared in a 1/1 ratio; GA-OSA 7/3=gum Arabic-OSA starch conjugate prepared in a 7/3 ratio; GA-OSA 9/1=gum Arabic-OSA starch conjugate prepared in a 9/1 ratio; GA-OSA 19/1=gum Arabic-OSA starch conjugate prepared in a 19/1 ratio; EM10=Super Gum EM10″ from San -Ei Gen F.F.I

At the highest modulus values (Gd=30-100 Pa), the rheological properties of the solution are mainly determined by the dissolved polymeric chains. A lower phase angle at a given value of the dynamic modulus indicates a broader molecular weight distribution or the presence of long chain branched molecules (Trinkle, S., and Freidrich, C. Rheologica Acta, (2001) 40 (4), pp. 322-328). The latter is e.g. the case for the GA-OSA 7/3 material. We defined the value of the phase angle at a fixed value of the dynamic modulus of 100 Pa as indicator for the width of the molecular weight distribution (PD) of the dissolved polymer. This value can be obtained by inter- or extrapolation of the delta vs. Gd plot on a linear-logarithmic scale.

PD GA<GA-OSA 1/1<GA CL<EM 10<GA-OSA 19/1<GA-OSA 9/1<GA-OSA 7/3

Additionally the phase angle at low values of the dynamic modulus can be used as an indicator for the presence of an elastic/gel-like fraction. In the case that a (permanent) elastic fraction is present, the phase angle shows a strong decrease with decreasing dynamic modulus. A lower value of the phase angle indicates the presence of more gel. We used the value of the phase angle at a dynamic modulus of 1 Pa as an indicator for the amount of gel present in the material, under the pre-requisite that the phase angle shows a decrease of more than 1 degree with decreasing dynamic modulus below 10 Pa. This results in the following ranking for the gel-fraction (GF):

GF EM10<GA<GA-OSA 1/1<GA CL

The other materials do not indicate from the rheological properties that a gel is present, because the phase angle does not decrease with decreasing dynamic modulus. The sample GA-OSA 7/3 does however show a much lower value of the phase angle at Gd=1 Pa, which is attributed to the presence of high molecular weight, probably long chain branched, polymeric chains.

### Conclusions

The results given in FIGS. 1 and 2 therefore demonstrate:
The presence of a small gel fraction in the Gum Arabic.
An increase of the molecular weight from Gum Arabic to Gum Arabic heated 48 hours (CL) in combination with a broadening of its molecular weight distribution and a significant increase of the gel fraction.
An increase of the molecular weight of the GA-OSA 19/1 and GA-OSA 9/1 plant gum—modified food starch conjugates in comparison with the Gum Acacia accompanied with a disappearance of the gel-fraction
An even more pronounced increase of the molecular weight of the GA-OSA 7/3 plant gum—modified food starch conjugate in combination with significant broadening of the molecular weight distribution, probably due to the presence of long-chain branched molecules. The presence of a gel fraction cannot be detected.
A narrower molecular weight distribution in the case of the GA-OSA 1/1 plant gum—modified food starch conjugate in comparison with the GA-OSA 19/1, GA-OSA 9/1 and GA-OSA 7/3 conjugates, in combination with the presence of a gel-like fraction. In comparison with GA and GA-CL this gel-fraction however has shorter relaxation times.
Finally, in comparison with GA, the EM 10 material has a higher molecular weight in combination with a broader molecular weight distribution like the conjugates. A very small gel-fraction is present, however much less than in the non-modified GA.

### Example 6

### Preparation of a Gum Acacia-OAS Starch Conjugate Containing Beta-Carotene

On a beaker, a gum based conjugate—gum acacia/OSA starch 1:1 was poured into water and stirred at room temperature until total dissolution of it. Meanwhile, the organic phase containing beta-carotene, corn oil, d-I α-tocopherol were dissolved into solvent Then, the organic phase was added to the aqueous phase and the resulting mixture emulsified (FLUID 5000 RPM). Finally, the solvent was evaporated under reduced pressure. The resulting product was converted into a dried form by powder catch (in a bed of starch).

TABLE 4
Gum acacia-OSA starch conjugate containing beta-carotene.

| Component | Amount (wt %) |
|---|---|
| Gum acacia-OSA starch conjugate | 58.75 |
| beta-carotene | 4.7 |
| dl-α-tocopherol | 1.42 |
| Cornoil | 5.12 |
| Powder catch | 30 |

### Example 7

### Preparation of a Gum Acacia-OSA Starch Conjugate Containing Tocopheryl Acetate

On a reactor, gum based conjugate (Gum acacia-OSA starch) and maltodextrin were successively poured into water and stirred at room temperature until total dissolution. After that, tocopheryl acetate previously warmed up to 60° C. was added to the aqueous phase. Then the resulting mixture was emulsified (FLUID 4000 RPM). Finally, the resulting product was converted into a dried form by spray drying.

TABLE 5
Gum acacia-OSA starch conjugate containing tocopheryl acetate.

| Component | Amount (wt %) |
|---|---|
| Gum acacia-OSA starch conjugate | 30 |
| DL-α-tocopheryl acetate | 50 |
| Maltodextrin | 19 |
| Silicon dioxide | 1 |

1. A plant gum-modified food starch conjugate consisting of
a) one or more plant gum(s); and
b) one or more modified food starch(s);
wherein a 30 wt % solution in water of said isolated plant gum—modified food starch conjugate at 25° C. has a phase angle of less than 85 degrees at a value of the dynamic modulus of 1 Pa, as determined with oscillatory rheological measurements.

2. Plant gum-modified food starch conjugate according to claim 1 characterized in that the gum is gum acacia variety Acacia senegal.

3. Plant gum-modified food starch conjugate according to claim 1 characterized in that the modified food starch is octenyl-succinate starch.

4. A composition comprising
a) 10 to 70 weight-% of plant gum-modified food starch conjugate according to claim 1; and
b) 0.1 to 60% of one or more fat-soluble active ingredient(s)

5. Composition according to claim 4, characterized in that the fat-soluble active ingredient(s) (one or more compounds) are selected from the group consisting of vitamin A, D, E, K and derivatives thereof; polyunsaturated fatty acids or esters thereof; lipophilic health ingredients; carotenoids; and flavoring or aroma substances as well as mixtures thereof.

6. Composition according to claim 4, characterized in that the fat-soluble active ingredient(s) (one or more compounds) are carotenoids, especially beta-carotene, lycopene, lutein, bixin, astaxanthin, apocarotenal, beta-apo-8′-carotenal, beta-apo-12′-carotenal, canthaxanthin, cryptoxanthin, citranaxanthin and/or zeaxanthin.

7. Composition according to claim 4 characterized in that the composition further comprises one or more adjuvants and/or excipients.

8. A process for the manufacture of a plant gum-modified food starch conjugate according to claim 1 which comprises the following steps:
I) suspending separately both plant gum and modified food starch into water;
II) optionally removing non dissolved material from the suspended modified food starch;
III) mixing the aqueous solution of plant gum of step I) to the aqueous solution of modified food starch prepared as in step I) and optionally II) in a ratio 1:1 to 20:1 and stirring the resulting mixture for about one hour;
IV) optionally incubating the mixture of step III) 1 to 24 hours at room temperature;
V) drying the mixture of step IV) to remove water and to produce a powder;
VI) heating the resulting powder of step V) at a temperature between 50 and 150° C. for 1 to 72 hours to form the plant gum-modified food starch conjugate.
X) Optionally, suspending plant gum-modified food starches conjugates in water
XI) Optionally, diafiltration of the aqueous solution through a 500 k Dalton membrane (HOLLOW FIBER—GE Healthcare) until the volume diluted (Vd) is at least greater or equal to 4.
XII) Drying the mixture of step VIII) to remove water and to produce a powder.

9. A process for the manufacture of a composition as claimed in claim 4 which comprises the following steps:
I) dispersing an appropriate amount of plant gum-modified food starch conjugate in an appropriate amount of water;
II) adding one or more fat-soluble active ingredient(s) to the solution produced in step I);
III) emulsifying the mixture of step II) with a conventional emulsification process known to the person skilled in the art.
IV) optionally drying the emulsion of step III)

10. A plant gum—modified food starch conjugate obtainable according to claim 8.

11. Use of a composition as claimed in claim 5 for the enrichment, fortification and/or coloration of food, beverages, animal feed, cosmetics or pharmaceutical compositions.

12. Process for the manufacture of a beverage by mixing a composition according to claim 5 with further usual ingredients of beverages.

13. Beverage obtainable by the process according to claim 12.
